(19) United States
(12) Patent Application Publication
(10) Pub. No.: US 2013/0017236 A1
(43) Pub. Date: Jan. 17, 2013

(54) Toothpaste or tooth gel containing silver nano particles coated with silver oxide

(71) Applicant: Robert Johnson Holladay, Saratoga Springs, UT (US)

(72) Inventor: Robert Johnson Holladay, Saratoga Springs, UT (US)

(21) Appl. No.: 13/135,575

(22) Filed: Jul. 11, 2011

(51) Int. Cl. A61K 8/04; A61Q 11/00; A61K 8/19

(52) U.S. Cl. 424/401; 424/49

(57) ABSTRACT

Toothpaste or tooth gel containing silver nano particles coated with silver oxide as the active ingredient.

### CROSS-REFERENCE TO RELATED APPLICATIONS

U.S. Pat. No. 7,135,195 describes the same silver nano particles coated with silver oxide used in a mouthwash. U.S. Pat. Nos. 6,214,299 and 6,743,348 describe a method of manufacturing the same silver nano particles coated with silver oxide.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention pertains to the dental field. Tooth decay has been linked to the presence of bacteria, and xylitol is a sugar that has antibacterial properties.

The silver nano particles coated with silver oxide used in this patent have been shown to have antibacterial action which is superior to other forms of colloidal silver, and are a composition of silver in water comprising a total concentration of silver of between about 0.0005-0.0040%, said silver in the form of colloidal silver particles having an interior of elemental silver and a surface of silver oxide, wherein a majority of the colloidal silver particles have a maximum diameter less than 0.015 micrometers, wherein a majority of the colloidal silver particles have a minimum diameter greater than 0.005 micrometers, and wherein the composition exhibits antimicrobial properties (U.S. Pat. No. 7,1335,195).

### BRIEF SUMMARY OF THE INVENTION

By mixing xylitol and silver nano particles in a toothpaste, a composition is formed which incorporates 2 different antibacterial agents, which act synergistically in eliminating bacteria.

While numerous previous toothpaste patents describe xylitol and different forms of silver, the addition of silver nano particles coated with silver oxide to toothpaste represents a novel invention.

### BRIEF DESCRIPTION OF THE SEVERAL VIEWS OF THE DRAWING

Not Applicable

### DETAILED DESCRIPTION OF THE INVENTION

The present invention consists of a toothpaste or tooth gel incorporating, as its active ingredient silver nano particles coated with silver oxide. A secondary active ingredient is xylitol. The antibacterial activity of silver oxide-coated silver nano particles such as those produced by using the apparatus and method disclosed in U.S. Pat. Nos. 6,214,299 and 6,743,348 has been amply demonstrated to remove or kill pathogenic microorganisms. Numerous studies have shown that xylitol has significant antibacterial properties. The synergistic or additive effect of these two ingredients may be greater than either applied separately. Additionally, the antibacterial and cleaning effects of these two ingredients has been shown to provide a favorable environment for tooth re-mineralization.

The present invention is designed to be used in the manner and frequency that is normally associated with toothpaste and tooth gels.

### Range of Ingredients:

Purified Water
xylitol (1-20%)
nano silver particles coated with silver oxide (0.0005-0.0040%)
Carbomer (0.5-1.5%)
Triethanolamine (0.5-2.5%)
Mint Flavoring (0.1-0.5%)
Food Coloring (0.05%)

### Preferred Embodiment:

Purified Water (87.1468%)
xylitol (10%)
nano silver particles coated with silver oxide (0.0032%)
Carbomer (1%)
Triethanolamine (1.5%)
Mint Flavoring (0.3%)
Food Coloring (0.05%)

### Method of Manufacture:

A suitable stainless steel jacketed kettle is obtained and is filled with a solution of purified water and silver oxide coated silver nano particles. The solution is heated to 120 degrees Fahrenheit and the carbomer (or other gelling agent) is added. This mixture is stirred for approximately one hour, or until the gelling agent is fully dissolved. The other ingredients (except triethanolamine) are added and the mixture is agitated until all agents are uniformly mixed throughout the solution. Triethanoloamine is then added and the mixture is agitated until the ingredients are mixed uniformly throughout the product. The mixture is then cooled and placed into tubes.

The form of the invention found in the preferred embodiment is intended to be typical and not limiting. Anyone skilled in the art can readily see that other colorings and flavorings and the addition of such items as silica and/or sodium fluoride could readily be part of the formulation without departing from the scope of the current invention. It is not known at this time if any of the ingredients other than silver oxide coated nano particles and xylitol contributes to the synergistic effect of the invention.

What is claimed is:

1. A toothpaste or tooth gel wherein the active ingredient is a composition of silver in water comprising a total concentration of silver of between about 0005-0.0040%, said silver in the form of colloidal silver particles having an interior of elemental silver and a surface of silver oxide, wherein a majority of the colloidal silver particles have a maximum diameter less than 0.015 micrometers, wherein a majority of the colloidal silver particles have a minimum diameter greater than 0.005 micrometers, and wherein the composition exhibits antimicrobial properties.
